(19) United States
(12) Patent Application Publication
(10) Pub. No.: US 2009/0223286 A1
(43) Pub. Date: Sep. 10, 2009

(54) ANALYTICAL METHOD FOR DETERMINATION OF RELATED SUBSTANCES OF IMIPENEM AND CILASTATIN

(76) Inventors: Vandana SINGH, Gurgaon (IN); Monish Chaddha, Gurgaon (IN); Sita Mahalakshmi, Gurgaon (IN); Sunil Yadav, Gurgaon (IN); Rita Santhakumar, Gurgaon (IN); T.G. Chandrashekhar, Gurgaon (IN)

Correspondence Address: RANBAXY INC.;INTELLECTUAL PROPERTY DEPT., 600 COLLEGE ROAD EAST, SUITE 2100, PRINCETON, NJ, 08540, US

(21) Appl. No.: 12/371,143

(22) Filed: Feb. 13, 2009

(30) Foreign Application Priority Data
Feb. 13, 2008 (IN) 378/DEL/2008

(51) Int. Cl. G01N 30/02

(52) U.S. Cl. 73/61.52

(57) ABSTRACT

A HPLC based analytical method for identification and quantification of related substances of imipenem, cilastatin and their combination is disclosed.

### FIELD OF THE INVENTION

The technical field of the present invention relates to a HPLC based analytical method for identification and quantification of related substances of imipenem and cilastatin and their combination.

### BACKGROUND OF THE INVENTION

Pharmaceutical products are regulated in most countries by a government agency such as the U.S. Food & Drug Administration (USFDA). These agencies generally require an applicant to show safety and efficacy of the pharmaceutical product during the review-approval phase and continue to monitor the safety of the drug post-approval. In order to satisfy the safety concerns, the regulatory agencies generally require a manufacturing specification that sets the maximum amount of each identified impurity, termed as “related substance”, as well as the maximum amount for all remaining unidentified impurities. Once approved, each batch or lot of the pharmaceutical product is tested to insure that the approved specifications are met. Further, stability testing is performed on the pharmaceutical product in order to show that the composition does not substantially or materially change over time; i.e., over its indicated shelf-life. Good practice warrants keeping samples from every commercial batch released to the public, so that the stability can be monitored and any defect uncovered and corrected.

Accordingly, pharmaceuticals i.e., both the active pharmaceutical ingredient and the finished product are tested for purity during manufacturing and subsequently during its shelf life. Typically, the product is tested by comparing certain analytical test results with those of a standard reference results. For detection of related substances, this normally requires assaying both the pharmaceutical product and pure related substances using validated analytical methods and comparing the results for qualitatively and quantitatively characterizing the product.

Heretofore, in the art there are various analytical tools available for the above characterization of products. These tools form a part of the day to day analytical work of the pharmaceutical industry and include nuclear magnetic resonance (NMR), liquid or gaseous chromatography (LC or GC) techniques coupled with suitable detectors (absorption, fluorescence or mass detectors), Infra red (IR), and X-ray diffraction (XRD) techniques. Out of these, one of the most widely used, and highly accurate is the high performance liquid chromatography (HPLC) technique coupled with one or more detectors, as per the requirements.

Combination product of imipenem and cilastatin is marketed by Merck since 1987, under the trade name Primaxin®, as 250 mg or 500 mg of each component in a single vial with sodium bicarbonate 2.0% present (on a weight basis) as a buffer. The contents of the vials need to be diluted in suitable volumes of an infusion fluid and then administered intravenously to the patient. Commonly used diluents and infusion fluids include sterile water for injection, 0.9% sodium chloride injection, 5% dextrose injection, 10% dextrose injection, 5% dextrose and 0.225% sodium chloride injection, 5% dextrose and 0.45% sodium chloride injection, 5% dextrose and 0.9% sodium chloride injection, Normosol®-M in 5% dextrose injection, 5% dextrose and 0.02% sodium bicarbonate injection, 5% dextrose and 0.15% potassium chloride injection, M/6 sodium lactate injection, Ringer's injection-lactated, Ringer's injection-lactated with 5% dextrose, 5% sodium bicarbonate injection, 2.5% mannitol injection, 5% mannitol injection, and 10% mannitol injection.

The Journal article published in American Journal of Hospital Pharmacy, Vol. 43, 2803-09 (1986), by Bigley et al., describes a stability indicating HPLC analytical method, which can detect and quantify imipenem and its primary metabolite thienamycin, and cilastatin and its primary metabolite cilastatin sulfoxide. The HPLC method was used with Hewlett-Packard RP-8 column (20 cm×0.46 cm internal diameter) and the column oven set at 50° C.; the injector volume was 10 μl and the flow rate was 4 ml/min throughout. The eluted peaks were detected at 250 mm by a Hewlett-Packard variable wavelength detector attached to the chromatograph. The mobile phase consisted of 0.004 M 3-[n-morpholino]propanesulphonic acid (MOPS) buffer with sodium hexane sulphonate 2 g/L; the mobile phase was adjusted to pH 7.00 with sodium hydroxide. The run time was less than 6 minutes.

The USP monograph of “Imipenem and Cilastatin for Injection” suggests its assay using the liquid chromatograph with a 254 nm detector and using a mobile phase of sodium 1-hexanesulfonate with a pH 6.8 buffer. The injection should contain not less than 90.0 percent and not more than 115.0 percent of the labeled amounts of imipenem and cilastatin.

Further the journal article published in Journal of Pharmaceutical and Biomedical analysis, Vol. 11(6), 477-82 (1993), by Parra et al., discloses a UV spectrophotometric assay based on the first and second order spectrophotometry suitable for the quantification of mixtures of imipenem and cilastatin sodium injection.

Besides the above, a few other analytical methods are available in the art that can detect imipenem, cilastatin and very few of its related impurities. There is, however, a long felt need in the art for an analytical method that can detect and quantify related substances of imipenem, cilastatin, and their combinations.

### SUMMARY OF THE INVENTION

We have now developed a HPLC based analytical method that can identify and quantify wide range of related substances of imipenem, cilastatin and their combinations, formed at different time points in different reconstituting diluents and at different storage conditions. These methods can be used to detect the presence of related substances, purify the product if required by reported methods, and confirm that the imipenem cilastatin combination product is of acceptable pharmaceutical quality.

Hence, in one general aspect, there is provided a HPLC based analytical method for the identification and quantification of more than 25 related substances of imipenem, cilastatin, and their combinations.

In another general aspect, there is provided a HPLC based analytical method for identification and quantification of related substances of imipenem, cilastatin, and their combination, wherein the mobile phase comprises a gradient of three essential components a) basic buffer, b) acidic buffer, and c) organic solvent.

In another general aspect, there is provided a HPLC based analytical method for identification and quantification of related substance of imipenem, cilastatin, and their combination, wherein the mobile phase comprises a gradient of three essential components a) basic buffer having a pH of about 6.5 to about 8.5, b) acidic buffer having a pH of about 1.5 to about 3, and c) polar organic solvent.

In another general aspect, there is provided a HPLC based analytical method for identification and quantification of related substances of imipenem, cilastatin, and their combination, wherein the mobile phase comprises a gradient of three essential components:
a) basic buffer comprising ammonium phosphate, and having a pH of about 7.7,
b) acidic buffer comprising orthophosphoric acid and having a pH of about 2.25, and
c) acetonitrile.

In another general aspect there is provided a HPLC based analytical method for identification and quantification of related substances of imipenem, cilastatin, and their combination, wherein
i) the mobile phase comprises a gradient of three essential components a) basic buffer, b) acidic buffer, and c) organic solvent,
ii) flow rate of the mobile phase is about 1 mL/min to about 2 mL/min,
iii) total run time is about 100 minutes,
iv) chromatographic detector is a PDA detector set over a range of 200-400 nm or a dual wavelength detector set at 210 and 300 nm; and
v) column is Luna C-8(2), 5 μm (250 mm×4.6 mm).

### BRIEF DESCRIPTION OF THE DRAWINGS

FIG. 1 is a Prototype chromatogram obtained for control sample in 10% Dextrose.

FIG. 2 is a Prototype chromatogram obtained for control sample in 10% Dextrose.

### DETAILED DESCRIPTION OF THE INVENTION

The mobile phase gradient in the above aspects may be adjusted through the run time in such a way that at any point of time the mobile phase would comprise one or more of the three essential components.

In clinical practice, contents of imipenem and cilastatin vials is first diluted in one or more injectable fluids to the desired concentration and then administered intravenously to the patient at a suitable rate. These diluting injectable fluids include 0.9% w/v sodium chloride (normal saline), 5% or 10% w/v dextrose injection, 5% w/v dextrose and 0.225% w/v sodium chloride injection, 5% w/v dextrose and 0.15% w/v potassium chloride injection, 5% w/v dextrose and 0.9% w/v sodium chloride injection, 5% w/v dextrose and 0.45% w/v sodium chloride injection, 5% w/v dextrose and 0.02% w/v sodium bicarbonate injection, and 5% or 10% w/v mannitol injection. The analytical method is suitable for analysis of the product in any of the injection fluids. Further, the analytical method is suitable for identification and quantification of related substances of imipenem, cilastatin, and their combination, in very low concentration as evident from the limit of detection (LOQ) values provided below in Table 1.

TABLE 1
LOQ values

| S. No. | Name | LOQ (% w/w) |
|---|---|---|
| 1 | Imipenemoic acid-1 | 0.016 |
| 2 | Imipenemoic acid-2 | 0.025 |
| 3 | Thienamycin | 0.02 |
| 4 | 7-Cystein-2-oxoheptanoic acid | 0.042 |
| 5 | Impurity A | 0.017 |
| | (Cilastatin sulphoxide epimer 1) | |
| 6 | Impurity A | 0.012 |
| | (Cilastatin sulphoxide epimer 2) | |
| 7 | Imipenem-cilastatin adduct-1 | 0.016 |
| 8 | Imipenem-cilastatin adduct-2 | 0.032 |
| 9 | Decarboxylated cilastatin | 0.007 |
| 10 | Delta-3-cilastatin | 0.011 |

Imipenem is [5R-[5α,6α(R*)]]-6-(1-hydroxyethyl)-3-[[2-[(iminomethyl)amino]ethyl]thio]-7-oxo-1-azabicyclo[3.2.0]hept-2-ene-2-carboxylic acid. It is the N-formimidoyl derivative of thienamycin. It is the first member of a new class of beta-lactam antibiotics, the carbapenems, having a broad-spectrum activity against aerobic and anaerobic bacteria, which is partly due to its high stability in the presence of β-lactamases. Imipenem is inherently unstable in solution as well as sensitive to heat and light. On degradation it also forms few colored impurities, which make the appearance of imipenem as pale yellow to brownish instead of the desired white color. Further, imipenem is also susceptible to quick metabolism via hydrolysis by the renal brush border dehydropeptidase decreasing the therapeutic efficacy of the drug. In clinical practice, imipenem is thus administered in combination with cilastatin, a specific and highly active dehydropeptidase inhibitor thereby improving the plasma concentrations and efficacy of imipenem. Chemically cilastatin is [R-[R*,S*-(Z)]]-7-[(2-amino-2-carboxyethyl)thio]-2-[[(2,2-dimethylcyclopropyl)carbonyl]amino]-2-heptenoic acid. A sterile formulation of imipenem and cilastatin sodium, having sodium bicarbonate as buffer is available in the market under the trade name Primaxin® IV

The term “related substances”, as used herein, refers to any known or unknown compounds (impurities) present in the sample. The related substances may be produced due to degradation from imipenem, cilastatin, or diluting fluids; may be introduced in the sample as process impurity; or may be even of an unknown origin. Some of the related substances may reduce the in vivo efficacy of the drug. Others may be toxic in nature, and may be undesirable above certain acceptable limits. Thus, stability testing of the drug as well as proper characterization of the impurity profile is warranted throughout the shelf life of the product. The analytical method developed by the inventors was able to resolve and quantify more than 25 related substances, in particular more than 40 related substances. Related substances in concentrations of more than about 0.10% w/w were reported and those in concentrations lower than 0.10% w/w were disregarded. Related substances that may be resolved include thienamycin, S (+) 2,2-dimethyl cyclopropane carboxamide, Impurity A (epimer 1 and 2), Impurity B, Impurity C, sodium carboxamido heptenoate, decarboxylated cilastatin, Delta-3-cilastatin, E isomer, methyl ester of cilastatin, and specified impurities of molecular weights 185, 317, 419, 343, 315, 305, 344, 479, 403, 263, 481, 479, 481, 403, 300, 598, 554, 319, 657, 356, 358, 744, 629, and 726.

The analytical method to carry out detection and quantification of imipenem, cilastatin, and the related substances is based on HPLC technique comprising a column, a suitable detector, and mobile phase.

All the other accessories necessary for carrying out analysis on a HPLC system are well known to any skilled analyst, and are thus assumed to be a part of the general state of art, and not being discussed separately.

HPLC system may be provided with the modern day softwares that can carry out accurate and reproducible analytical procedures. Further, the system should be able to extract and resolve the chromatograms, integrate the peak areas with precision and accuracy. One such HPLC system is available from Waters.

Related substances of imipenem, cilastatin, and their combination comprise chromophoric groups and can thus be detected using a detector based on absorption principle. The detector may be a dual wavelength detector set at 210 and 300 nm, or a Photo Diode Array (PDA) detector (such as 2996 PDA diode array detector from Waters) set over the range of 200-400 nm. Imipenem peak may be detected at 300 nm whereas that of cilastatin at 210 mm.

The column used may be any suitable HPLC column, which can resolve imipenem, cilastatin, and the related compounds. One such column that may be used is Luna C18 (2), 5 μm (250 mm×4.6 mm) from Phenomenex. The column may be provided with a temperature control apparatus with the temperature being set at about 20° C. to about 30° C., in particular about 30° C.

The mobile phase selection is one of the most critical factors of the analytical method and comprises of at least three essential components; a basic buffer (Buffer A), an acidic buffer (Buffer B), and an organic phase. Buffer A comprises of a phosphate component, particularly ammonium phosphate. It may also comprise alkali salt of perchloric acid, in particular sodium perchlorate. The pH of the buffer is adjusted to about 6.5 to about 8.5 using an anine particularly dimethylamine. pH adjustment is very critical and hence no acid should be used to lower the pH if the above limit is crossed, a fresh buffer should be prepared instead. In particular the pH of Buffer A is about 7.7. Buffer B comprises of a strong acid component, in particular orthophosphoric acid. The pH of the buffer is adjusted to about 1.5 to about 3.0 using a suitable acid or base, in particular sodium hydroxide or orthophosphoric acid. In particular the pH of Buffer B is about 2.25. The organic phase comprises a suitable organic solvent, in particular polar organic solvent such as acetonitrile. Buffer A, Buffer B, and the organic phase may be filtered through 0.22 μm membrane filter, if desired. The mobile phase is a combination of the three essential components in different ratios (by volume) over the entire run time period. Imipenem being soluble and stable in the basic pH, it is eluted first with the basic Buffer A, followed by an increase in the Buffer B and organic phase components to elute cilastatin and other related substances. The total run time of the mobile phase is about 100 minutes, with imipenem and cilastatin peaks appearing at 11 and 50 minutes respectively. Peaks of all the other related substances are spread over the chromatogram in the entire run time range. The flow rate of the mobile phase may be adjusted from about 1 mL/min to about 2 mL/min, particularly 1.5 mL/min.

In order to further illustrate the present invention and the advantages thereof, a detailed prototype analysis method is provided below. However, the prototype is for the purpose of illustration and should not be construed as limiting on the scope of the present invention.

### Preparation of Mobile Phase

### Preparation of Buffer A

1.15 g of ammonium phosphate monobasic and 0.5 g of sodium perchlorate monohydrate were transferred in 1 L of distilled water and dissolved. The pH of the solution was adjusted with dimethylamine to 7.7±0.02 and filtered through 0.22 μm membrane filter to form Buffer A.

### Preparation of Buffer B

2 mL of orthophosphoric acid was added to 1 L of distilled water and dissolved. The pH of the solution was adjusted with 10% w/v sodium hydroxide solution or 10% v/v orthophosphoric acid solution to 2.25±0.02 and filtered through 0.22 μm membrane filter to form Buffer B.

Note: Accurate pH adjustment is very critical for separation.

### Preparation of Organic Phase

Acetronitrile was used as the organic phase.

### Preparation of Diluent (Solution)

1.36 g of potassium dihydrogen phosphate was transferred to 1 L of distilled water and dissolved. The pH of the solution was adjusted with 10% w/v potassium hydroxide solution to 6.8±0.05 to form the diluent. The temperature of the diluent was maintained between about 17° C. to about 22° C. throughout the time of sample and placebo preparation.

### Preparation of System Suitability Solution

50 mg of imipenem monohydrate working standard was accurately weighed and transferred to 100 mL volumetric flask. It was dissolved in 60 mL diluent and volume made up with the same. 5 mL of this solution was diluted to 50 mL with diluent. 10 mL of the resultant solution was further diluted to 25 mL with the diluent. The final solution was filtered through 0.45 μm nylon filter (make Millipore) and had a concentration of 0.02 mg/mL.

### Preparation of Standard Solution

### Stock Solution A′

50 mg of imipenem monohydrate working standard and 50 mg of cilastatin working standard were accurately weighed and transferred to 100 mL volumetric flask. They were dissolved in 60 mL diluent by sonicating in ice-cold water (not exceeding 15° C.), and volume made up with the same. 5.0 mL of this solution was diluted to 50 ml, with diluent to form Stock Solution A′ having a concentration of both imipenem monohydrate and cilastatin as 0.05 mg/mL.

### Standard Solution:

0.5 mg of decarboxylated cilastatin impurity was accurately weighed and transferred to 25 mL volumetric flask. It was dissolved in 5 mL of diluent by shaking. 10 mL of Stock Solution A′ prepared above was added to the flask, and volume made up with diluent. The final solution obtained was filtered through 0.45 μm nylon filter to form Stock Solution A′, having the concentration of imipenem monohydrate, cilastatin, and decarboxylated cilastatin impurity as 0.02 mg/mL.

Note: Filtered standard solution is stable up to 18 hours in cooling module at 5° C.

### Preparation of Sample Solution

### Stock Solution B

A small volume of diluent was added in the sample vial (imipenem and cilastatin injection vial), shaken and the contents transferred to a 100 mL volumetric flask. The above procedure was repeated for about 5-6 times to ensure complete transfer of vial contents to the volumetric flask. The volume of the reconstituting diluent in the volumetric flask was adjusted to about 95 mL, and agitated to get a clear solution. The volume was made up with the same diluent and mixed well to get Stock Solution B.

10 mL (for 500 mg strength) or 20 mL (for 250 mg strength) of Stock Solution B was transferred to 25 mL volumetric flask and volume made up with diluent. The final solution was filtered through 0.45 μm nylon filter and had the concentrations of imipenem monohydrate and cilastatin as 2 mg/mL. The sample solutions were fleshly prepared from the stock solution and injected immediately.

### Preparation of Placebo Solutions

### Common Placebo

The vial containing placebo (containing neither imipenem nor cilastatin) was constituted for both 500 mg and 250 mg strengths, in the same way as the preparation of sample solution.

### Imipenem Placebo

The vial containing placebo (containing cilastatin and not imipenem) was constituted for both 500 mg and 250 mg strengths, in the same way as the preparation of sample solution.

### Cilastatin Placebo

The vial containing placebo (containing imipenem and not cilastatin) was constituted for both 500 mg and 250 mg strengths, in the same way as the preparation of sample solution. The cilastatin placebo was freshly prepared and injected immediately.

### Preparation of Spiked Impurity Sample Solution

0.5 mg of each of the available impurities of cilastatin were accurately weighed and transferred to a 10 mL volumetric flask. 3 mL of diluent was added and sonicated to dissolve the impurities. The volume was made up with diluent to prepare the Impurity Stock Solution.

50 mg of cilastatin working standard was accurately weighed and transferred to a 25 mL volumetric flask. It was dissolved by sonication in 15 mL of diluent. 2 mL of Impurity Stock Solution was added and the volume made up with the diluent. The final solution was filtered through 0.45 μm nylon filter and had the concentrations of cilastatin and the impurities as 2 mg/mL and 0.004 mg/mL respectively.

Imipenem impurities were not added to the spiked sample, as they are not stable in solution.

### Time Points of Analysis

The sample and placebos were analyzed at three different time points 0 hours, 4 hours (maintained at 24° C.±0.5° C.) and 24 hours (maintained at 3° C.±0.5° C.). For 0 hours, samples and placebos were diluted immediately from the stock solutions and for further time points, they were prepared from stock solutions stored accordingly. Imipenem placebo was not analyzed at 4 hours as the placebo solution is stable and for the identification of impurities related to cilastatin, 0 hours imipenem placebo chromatogram was referred.

### Chromatographic Parameters

The chromatographic parameters were as follows:

| | |
|---|---|
| Column | Luna C18 (2), 5 μm (250 mm × 4.6 mm) |
| Column oven temperature | 30° C. |
| Mobile phase flow rate | 1.5 mL/min |
| Detector | Dual wavelength - UV at 210 nm and 300 |
| | nm or PDA - Wavelength range 200-400 nm |
| Sample tray temperature | 5° C. |
| Injection volume | 20 μL |
| Run time | 100 minutes |
| Injection delay time | 10 minutes |
| Needle wash | Set double needle wash option |

### Mobile Phase Gradient

The mobile phase gradient over the total run time of 100 minutes was as follows:

| Time (min) | Buffer A | Buffer B | Organic phase |
|---|---|---|---|
| 0 | 100 | 0 | 0 |
| 15 | 100 | 0 | 0 |
| 40 | 10 | 80 | 10 |
| 65 | 0 | 82 | 18 |
| 70 | 0 | 82 | 18 |
| 80 | 0 | 65 | 35 |
| 90 | 65 | 0 | 35 |
| 100 | 100 | 0 | 0 |

### Analysis Procedure

### Column Washing and Equilibrium

Before starting the analysis column was washed with acetonitrile and water in the ratio of 50:50. for about 15 minutes, followed by equilibration for 20 minutes. with initial gradient of the mobile phase. After the completion of the analysis, the column was washed by running a mixture of acetronitrile and water as per the gradient given below:

| Time (minutes) | Acetonitrile | Water |
|---|---|---|
| 0 | 10 | 90 |
| 40 | 10 | 90 |
| 50 | 50 | 50 |
| 100 | 50 | 50 |
| 110 | 100 | 0 |
| 140 | 100 | 0 |
| 150 | 50 | 50 |

### Evaluation of System Suitability

5 replicate injections of system suitability solution and duplicate injections of standard solution were injected into the chromatograph. The chromatogram of system suitability solution was extracted at 300 nm and that of standard solution at both 210 and 300 nm.

The system was considered to be suitable for analysis only if the following conditions were met:
Column efficiency determined for imipenem peaks at 300 nm, was not less than 6000 theoretical plates.
The tailing factor for imipenem peaks at 300 nm was not less than 0.7 and not more than 1.5.
The relative standard deviation for five replicate injections for imipenem peak at 300 nm, was not more than 2%.
The resolution between cilastatin and decarboxylated cilastatin peaks was not less than 7.0, in the chromatogram of standard solution.

### Sample Analysis and Evaluation of the Chromatograms

After system suitability evaluation, sample solution and placebo solutions were injected into the system and the chromatograms extracted at 210 and 300 nm.

The chromatograms were extracted at 210 nm and 300 nm (except for imipenem placebo which was extracted only at 210 nm). The retention time of the imipenem peak was about 11 minutes and that of cilastatin was about 50 minutes.

The common placebo chromatogram was examined for any extraneous peaks, and the corresponding peaks disregard (if observed) in the chromatogram of the sample solution and in the chromatograms of the imipenem and cilastatin placeboes. Generally, the placebo peaks should have a retention time different than that of the known impurities.

The chromatogram of imipenem placebo was examined to identify all the impurities of cilastatin in the chromatogram of the sample solution. These impurities were quantified against cilastatin peak area, as obtained in standard solution chromatogram at 210 nm.

The chromatogram of cilastatin placebo was examined both at 210 and 300 nm to identify all the impurities of imipenem in the chromatogram of the sample solution both at 210 and 300 nm. Impurities specified at 300 nm were quantified against imipenem peak area, as obtained in standard solution chromatogram at 300 nm; and those specified at 210 nm against imipenem peak area, as obtained in standard solution chromatogram at 210 nm.

Any other known impurities (impurities not observed in imipenem and cilastatin placebo i.e., their origin is not known) were quantified against imipenem peak area, as obtained in standard solution chromatogram at 210 nm.

Any other unknown impurities (impurities not observed in imipenem and cilastatin placebo i.e., their origin is not known) were examined at both the wavelengths (210 nm and 300 nm) and higher values at either of these wavelength were considered. If higher values were observed at 210 nm then quantification was done against imipenem peak area, as obtained in standard solution chromatogram at 210 nm and if higher level were observed at 300 mm than quantification was done against imipenem peak area, as obtained in standard solution chromatogram at 300 nm.

Note: Reject any peaks in the sample due to mesityl oxide at the retention time of about 53.7 (RRT²≅1.06 wrt cilastatin).

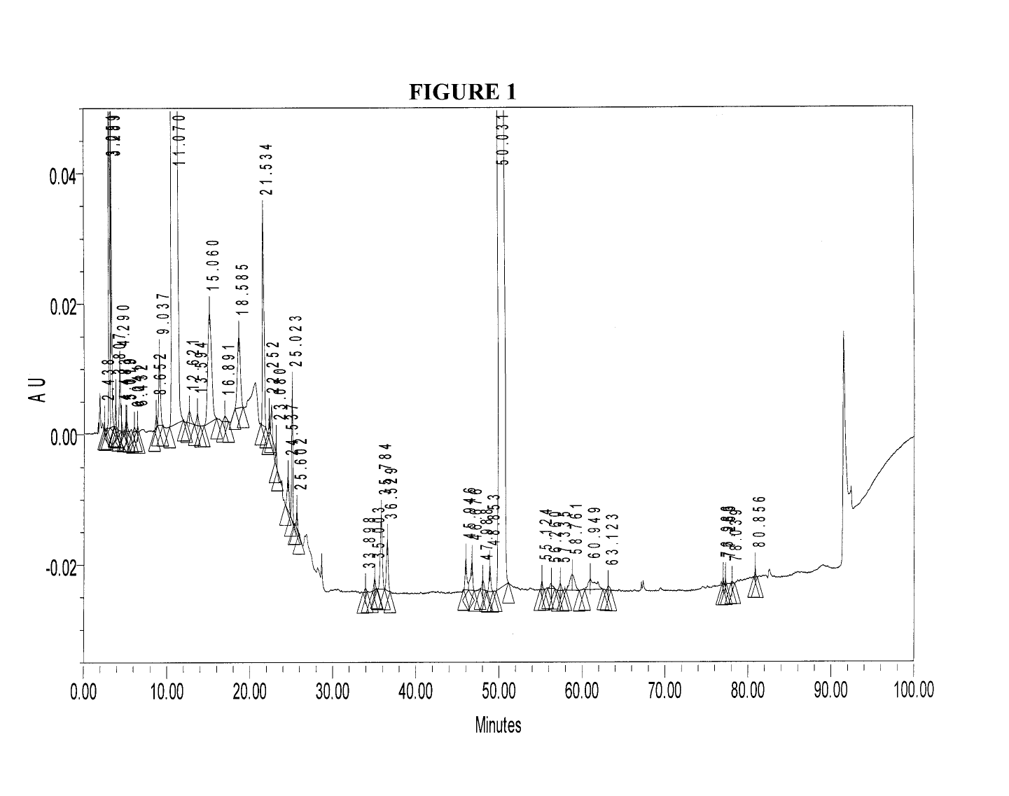

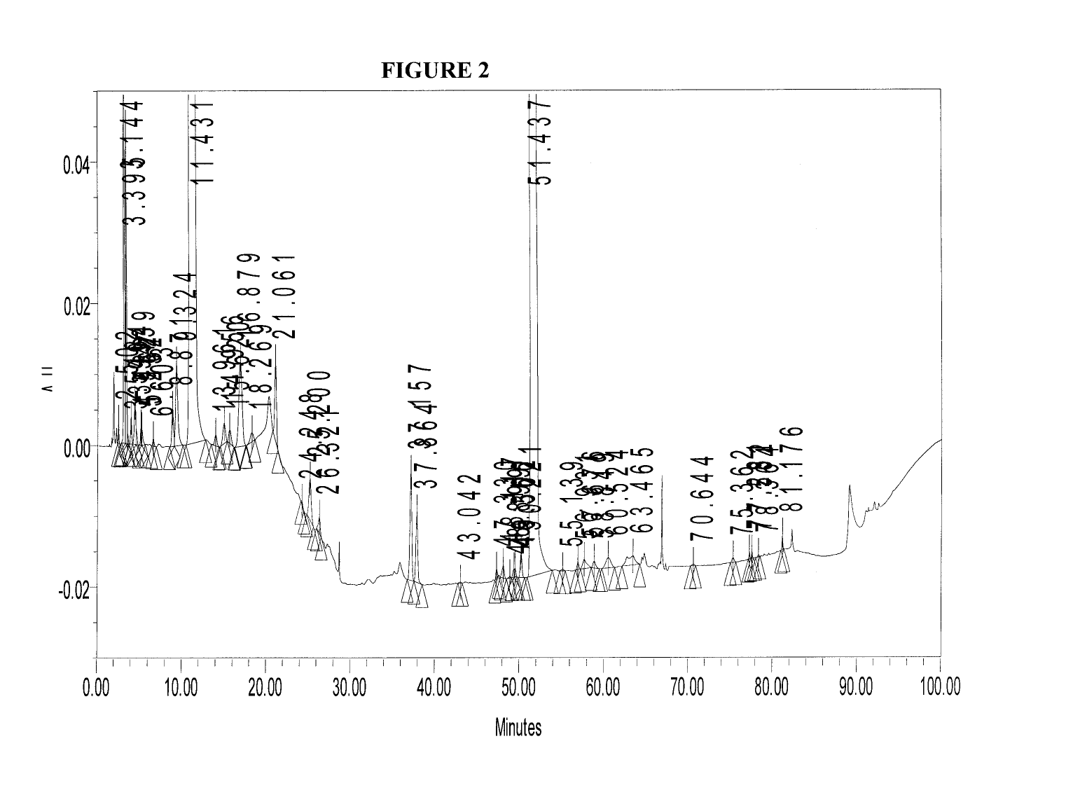

The relative retention times (RRT's) of the impurities is represented below in Table 2, and representative chromatograms are provided in FIG. 1 (control sample in 10% dextrose injection) and FIG. 2 (control sample in 10% mannitol injection). In general, the relative retention times (RRTs) for the impurities prior to Impurity A were calculated wrt imipenem peak, and RRTs for impurities from Impurity A onwards were calculated wrt. cilastatin peak. In the chromatograms of the sample solutions at 300 nm RRT's were calculated wrt. imipenem peak.

TABLE 2
Relative Retention Times (RRT's)

| | | RRT¹ | RRT¹ | RRT² | RRT² | | | |
|---|---|---|---|---|---|---|---|---|
| | | (for all | (for | (for all | (for | | | |
| | | other | Mannitol | other | Mannitol | Source of | Wavelength | |
| No. | Name | diluents) | only) | diluents) | only) | Impurity | (nm) | Origin |
| 1 | Specified Impurity | ≃0.23 | ≃0.23 | — | — | I | 300 | D |
| | (MW 185) at | | | | | | | |
| | RRT ≃0.23 | | | | | | | |
| 2 | Imipenemoic acid- | ≃0.28 | ≃0.28 | — | — | I | 210 | P/D |
| | 1 | | | | | | | |
| 3 | Imipenemoic acid- | ≃0.30 | ≃0.30 | — | — | I | 210 | P/D |
| | 2 | | | | | | | |
| 4 | Specified impurity | ≃0.35 | ≃0.35 | — | — | I | 210 | D |
| | (MW 419) at | | | | | | | |
| | RRT ≃0.35 | | | | | | | |
| 5 | Specified Impurity | ≃0.39 | ≃0.39 | — | — | I | 210 | D |
| | (MW 343) at | | | | | | | |
| | RRT ≃0.39 | | | | | | | |
| 6 | Specified Impurity | ≃0.45 | ≃0.45 | — | — | I | 210 | P/D |
| | (MW 315) at | | | | | | | |
| | RRT ≃0.45 | | | | | | | |
| 7 | Specified Impurity | ≃0.46 | ≃0.46 | — | — | I | 210 | P/D |
| | (MW 315) at | | | | | | | |
| | RRT ≃0.46 | | | | | | | |
| 8 | Specified impurity | ≃0.51 | ≃0.51 | — | — | I | 300 | P/D |
| | (MW 305) at | | | | | | | |
| | RRT ≃0.51 | | | | | | | |
| 9 | Specified Impurity | ≃0.77 | ≃0.77 | — | — | I | 210 | D |
| | (MW 344) at | | | | | | | |
| | RRT ≃0.77 | | | | | | | |
| 10 | Thienamycin | ≃0.82 | ≃0.82 | — | — | I | 300 | P/D |
| 11 | Specified impurity | ≃1.12 | — | — | — | I | 210 | D |
| | (MW 479) at | | | | | | | |
| | RRT ≃1.12 | | | | | | | |
| 12 | Specified impurity | — | ≃1.19 | — | — | I | 210 | D |
| | (MW 403) at | | | | | | | |
| | RRT ≃1.19 | | | | | | | |
| 13 | 7-Cystein-2- | ≃1.21 | ≃1.21 | — | — | C | 210 | P/D |
| | oxoheptanoic acid | | | | | | | |
| 14 | Specified impurity | — | ≃1.34 | — | — | I | 210 | D |
| | (MW 481) at | | | | | | | |
| | RRT ≃1.34 | | | | | | | |
| 15 | Specified impurity | ≃1.36 | — | — | — | I | 210 | D |
| | (MW 479) at | | | | | | | |
| | RRT ≃1.36 | | | | | | | |
| 16 | Specified impurity | — | ≃1.37 | — | — | I | 210 | D |
| | (MW 481) at | | | | | | | |
| | RRT ≃1.37 | | | | | | | |
| 17 | Specified impurity | — | ≃1.49 | — | — | I | 210 | D |
| | (MW 481) at | | | | | | | |
| | RRT ≃1.49 | | | | | | | |
| 18 | Specified Impurity | ≃1.55 | — | — | — | I | 210 | — |
| | (MW 479) at | | | | | | | |
| | RRT ≃1.55 | | | | | | | |
| 19 | Specified Impurity | — | ≃1.59 | — | — | I | 210 | — |
| | (MW 481) at | | | | | | | |
| | RRT ≃1.59 | | | | | | | |
| 20 | Specified impurity | ≃1.64 | — | — | — | I | 210 | D |
| | (MW 479) at | | | | | | | |
| | RRT ≃1.64 | | | | | | | |
| 21 | Specified impurity | — | ≃1.74 | — | — | I | 210 | D |
| | (MW 481) at | | | | | | | |
| | RRT ≃1.74 | | | | | | | |
| 22 | Specified impurity | — | ≃1.85 | — | — | I | 210 | D |
| | (MW 481) at | | | | | | | |
| | RRT ≃1.85 | | | | | | | |
| 23 | Specified impurity | ≃1.93 | — | — | — | I | 210 | D |
| | (MW 479) at | | | | | | | |
| | RRT ≃1.93 | | | | | | | |
| 24 | Specified impurity | ≃1.99 | — | — | — | I | 210 | — |
| | (MW 479) at | | | | | | | |
| | RRT ≃1.99 | | | | | | | |
| 25 | Specified impurity | ≃2.17 | ≃2.17 | — | — | I | 210 | D |
| | (MW 403) at | | | | | | | |
| | RRT ≃2.17 | | | | | | | |
| 26 | Specified impurity | ≃2.21 | ≃2.21 | — | — | I | 300 | D |
| | (MW 300) at | | | | | | | |
| | RRT ≃2.21 | | | | | | | |
| 27 | Specified impurity | ≃2.26 | — | — | — | I | 210 | D |
| | (MW 479) at | | | | | | | |
| | RRT ≃2.26 | | | | | | | |
| 28 | Specified impurity | ≃2.29 | — | — | — | I | 210 | — |
| | (MW 403) at | | | | | | | |
| | RRT ≃2.29 | | | | | | | |
| 29 | Specified impurity | ≃2.48 | ≃2.48 | — | ≃2.48 | I | 300 | D |
| | (MW 598) at | | | | | | | |
| | RRT ≃2.48 | | | | | | | |
| 30 | S(+)2,2-Dimethyl | ≃3.14 | ≃3.14 | — | ≃3.14 | C | 210 | P/D |
| | cyclopropane | | | | | | | |
| | carboxamide | | | | | | | |
| 31 | Specified impurity | ≃3.00 | ≃3.00 | — | ≃3.00 | I | 300 | P/D |
| | (MW 554) at | | | | | | | |
| | RRT ≃3.00 | | | | | | | |
| 32 | Impurity A | — | — | ≃0.73 | ≃0.73 | C | 210 | P/D |
| | (cilastatin | | | | | | | |
| | sulphoxide | | | | | | | |
| | Epimer 1) | | | | | | | |
| 33 | Impurity A | — | — | ≃0.75 | ≃0.75 | C | 210 | P/D |
| | (cilastatin | | | | | | | |
| | sulphoxide | | | | | | | |
| | Epimer 2) | | | | | | | |
| 34 | DMCA | — | — | ≃0.80 | ≃0.80 | C | 210 | P |
| | sulfoheptenoic | | | | | | | |
| | acid | | | | | | | |
| 35 | Imipenem- | — | — | ≃0.90 | ≃0.90 | — | 210 | D |
| | cilastatin adduct-1 | | | | | | | |
| 36 | Imipenem- | — | — | ≃0.91 | ≃0.91 | — | 210 | D |
| | cilastatin adduct-2 | | | | | | | |
| 37 | Specified Impurity | — | — | ≃0.96 | ≃0.96 | C | 210 | P |
| | (MW 356) at | | | | | | | |
| | RRT ≃0.96 | | | | | | | |
| 38 | Opened | — | — | ≃0.98 | ≃0.98 | C | 210 | P |
| | cyclopropyl | | | | | | | |
| | analogue of | | | | | | | |
| | Cilastatin | | | | | | | |
| 39 | Decarboxylated | — | — | ≃1.08 | ≃1.08 | C | 210 | P/D |
| | cilastatin | | | | | | | |
| 40 | Delta-3-cilastatin | — | — | ≃1.12 | ≃1.12 | C | 210 | P/D |
| 41 | Specified Impurity | — | — | ≃1.13 | — | — | 210 | — |
| | (MW 385) at | | | | | | | |
| | RRT ≃1.13 | | | | | | | |
| 42 | E isomer of | — | — | ≃1.14 | ≃1.14 | C | 210 | P |
| | cilastatin | | | | | | | |
| 43 | Specified impurity | — | — | ≃1.17 | ≃1.17 | — | 210 | D |
| | (MW 657) at | | | | | | | |
| | RRT ≃1.17 | | | | | | | |
| 44 | Specified impurity | — | — | ≃1.19 | ≃1.19 | — | 210 | D |
| | (MW 744) at | | | | | | | |
| | RRT ≃1.19 | | | | | | | |
| 45 | Methyl ester of | — | — | ≃1.19 | ≃1.19 | C | 210 | P |
| | cilastatin | | | | | | | |
| 46 | Specified impurity | — | — | ≃1.25 | ≃1.25 | — | 210 | D |
| | (MW 629) at | | | | | | | |
| | RRT ≃1.25 | | | | | | | |
| 47 | Impurity B | — | — | ≃1.27 | ≃1.27 | C | 210 | P |
| 48 | Impurity C | — | — | ≃1.37 | ≃1.37 | C | 210 | P |
| 49 | Specified impurity | — | — | ≃1.58 | ≃1.58 | — | 210 | D |
| | (MW 726) at | | | | | | | |
| | RRT ≃1.58 | | | | | | | |
| 50 | Sodium | — | | ≃1.72 | ≃1.72 | C | 210 | P |
| | carboxamido | | | | | | | |
| | heptenoate | | | | | | | |

*RRT¹ = Relative retention time wrt Imipenem, RRT² = Relative retention time wrt Cilastatin, I = Imipenem related, C = Cilastatin related, P = Process impurity, D = Degradation impurity

When the sample was reconstituted in dextrose diluents, the specified impurity MW 479 was observed. However, this impurity was absent on reconstitution in mannitol (5% and 10% w/v).

Specified impurity (MW 744) is a degradation impurity and was observed in 4 hours and 24 hours samples. This impurity co-elutes with methyl ester of cilastatin. However, this impurity was absent in 0 hours samples, therefore methyl ester of cilastatin was confirmed to be eluted at RRT²≅1.19. Further, methyl ester of cilastatin being a process impurity should remain constant, and the difference in the peak areas at different time points was used in quantification of this specified impurity.

In case of doubts regarding the retention times of the eluting peaks of the impurities, the same may be confirmed by injecting the spiked impurity sample solution, prepared by the procedure given above.

### Calculations

Impurities of Imipenem at 210 nm were calculated as follows:

$$\begin{matrix}Any individual known impurity of imipenem ( \% w \text{/} w ) ( \begin{matrix}\begin{matrix}except \\ process\end{matrix} \\ impurities\end{matrix} ) = \frac{AT}{{AS}_{1}} \times \frac{DS}{DT} \times \frac{P}{100} \times \frac{100}{C} & ( i ) \\ Highest unknown impurity of imipenem ( \% w \text{/} w ) = \frac{{AT}_{1}}{{AS}_{1}} \times \frac{DS}{DT} \times \frac{P}{100} \times \frac{100}{C} & ( ii ) \\ Total unknown impurities of imipenem ( \% w \text{/} w ) = \frac{{AT}_{2}}{{AS}_{1}} \times \frac{DS}{DT} \times \frac{P}{100} \times \frac{100}{C} & ( iii ) \\ Total known impurities = Sum of all known impurities ( except process impurities ) of imipenem & ( iv )\end{matrix}$$

Impurities of Imipenem at 300 nm were calculated as follows:

$$\begin{matrix}Any individual known impurity of imipenem ( \% w \text{/} w ) ( \begin{matrix}\begin{matrix}except \\ process\end{matrix} \\ impurities\end{matrix} ) = \frac{{AT}_{3}}{{AS}_{2}} \times \frac{DS}{DT} \times \frac{P}{100} \times \frac{100}{C} & ( v ) \\ Highest unknown impurity of imipenem ( \% w \text{/} w ) = \frac{{AT}_{4}}{{AS}_{2}} \times \frac{DS}{DT} \times \frac{P}{100} \times \frac{100}{C} & ( vi ) \\ Total unknown impurities of imipenem ( \% w \text{/} w ) = \frac{{AT}_{5}}{{AS}_{2}} \times \frac{DS}{DT} \times \frac{P}{100} \times \frac{100}{C} & ( vii ) \\ Total known impurities = Sum of all known impurities ( except process impurities ) of imipenem & ( viii )\end{matrix}$$

Impurities of cilastatin at 210 nm were calculated as follows:

$$\begin{matrix}Any individual known impurity of cilastatin ( \% w \text{/} w ) ( \begin{matrix}\begin{matrix}except \\ process\end{matrix} \\ impurities\end{matrix} ) = \frac{{AT}_{6}}{{AS}_{3}} \times \frac{{DS}_{1}}{DT} \times \frac{{P}_{1}}{100} \times \frac{100}{{C}_{1}} & ( ix ) \\ Highest unknown impurity of cilastatin ( \% w \text{/} w ) = \frac{{AT}_{7}}{{AS}_{3}} \times \frac{{DS}_{1}}{DT} \times \frac{{P}_{1}}{100} \times \frac{100}{{C}_{1}} & ( x ) \\ Total unknown impurities of cilastatin ( \% w \text{/} w ) = \frac{{AT}_{8}}{{AS}_{3}} \times \frac{{DS}_{1}}{DT} \times \frac{{P}_{1}}{100} \times \frac{100}{{C}_{1}} & ( xi ) \\ Total known impurities = Sum of all known impurities ( except process impurities ) of cilastatin & ( xii )\end{matrix}$$

Any Other Impurities at 210 nm were calculated as follows:

$$\begin{matrix}Any other known ( \% w \text{/} w ) = \frac{{AT}_{9}}{{AS}_{1}} \times \frac{DS}{DT} \times \frac{{P}_{1}}{100} \times \frac{100}{{C}_{2}} & ( xiii ) \\ Highest any other unknown impurity ( \% w \text{/} w ) = \frac{{AT}_{10}}{{AS}_{1}} \times \frac{DS}{DT} \times \frac{P}{100} \times \frac{100}{{C}_{2}} & ( xiv ) \\ Total any other unknown ( \% w \text{/} w ) = \frac{{AT}_{11}}{{AS}_{1}} \times \frac{DS}{DT} \times \frac{P}{100} \times \frac{100}{{C}_{2}} & ( xv ) \\ Total any other known = Sum of all any other known impurities & ( xvi )\end{matrix}$$

Any other impurities at 300 nm were calculated as follows:

$$\begin{matrix}Any other known impurity ( \% w \text{/} w ) = \frac{{AT}_{12}}{{AS}_{2}} \times \frac{DS}{DT} \times \frac{P}{100} \times \frac{100}{{C}_{2}} & ( xvii ) \\ Highest any other unknown impurity ( \% w \text{/} w ) = \frac{{AT}_{13}}{{AS}_{2}} \times \frac{DS}{DT} \times \frac{P}{100} \times \frac{100}{{C}_{2}} & ( xviii ) \\ Total any other unknown ( \% w \text{/} w ) = \frac{{AT}_{14}}{{AS}_{2}} \times \frac{DS}{DT} \times \frac{P}{100} \times \frac{100}{{C}_{2}} & ( xix ) \\ Total any other known = Sum of all any other known impurities & ( xx )\end{matrix}$$

### Where

AT=Area counts of known impurity peak (except process impurities) in the chromatogram of the sample solution at 210 nm.
AT₁=Area counts of highest unknown impurity peak in the chromatogram of the sample solution at 210 nm.
AT₂=Sum of area counts of unknown impurity peaks in the chromatogram of the sample solution at 210 nm.
AT₃=Area counts of known impurity peak (except process impurities) in the chromatogram of the sample solution at 300 nm.
AT₄=Area counts of highest unknown impurity peak in the chromatogram of the sample solution at 300 nm.
AT₅=Sum of area counts of unknown impurity peaks in the chromatogram of the sample solution at 300 mm.
AT₆=Area counts of known impurity peak (except process impurities) in the chromatogram of the sample solution at 210 nm.
AT₇=Area counts of highest unknown impurity peak in the chromatogram of the sample solution at 210 nm.
AT₈=Sum of area counts of unknown impurity peaks in the chromatogram of the sample solution at 210 nm.
AT₉=Area counts of any other known impurity in the chromatogram of the sample solution at 210 nm.
AT₁₀=Area counts of highest any other unknown impurity in the chromatogram of the sample solution at 210 nm.
AT₁₁=Sum of area counts of any other unknown impurity in the chromatogram of the sample solution at 210 nm.
AT₁₂=Area counts of any other known impurity in the chromatogram of the sample solution at 300 nm.
AT₁₃=Area counts of highest any other unknown impurity in the chromatogram of the sample solution at 300 μm.
AT₁₄=Sum of area counts of any other unknown impurity in the chromatogram of the sample solution at 300 nm.
AS₁=Average area counts of imipenem peak in the chromatogram of the standard solution at 210 nm.
AS₂=Average area counts of imipenem peak in the chromatogram of the standard solution at 300 nm.
AS₃=Average area counts of cilastatin peak in the chromatogram of the standard solution at 210 nm.
DS=Dilution factor of imipenem in the standard solution.
DS₁=Dilution factor of cilastatin in the standard solution.
DT=Dilution factor of the sample solution.
P=Percent potency of imipenem monohydrate working standard, as imipenem on as is basis.
P₁=Percent potency of cilastatin working standard, on as is basis.
C=Label claim of imipenem per vial in mg.
C₁=Label claim of cilastatin per vial in mg.
C₂=Mean of claim of imipenem and cilastatin per vial in mg.
Total impurities=(iii)+(iv)+(viii)+(xi)+(xii)+(xv)+(xvi)+(xix)+(xx)

We claim:

1. A HPLC based analytical method for the identification and quantification of more than 25 related substances of imipenem, cilastatin, and their combinations. Using a mobile phase comprising a gradient of three essential components a) basic buffer, b) acidic buffer, and c) organic solvent.

2. The HPLC based analytical method of claim 1 wherein a) basic buffer has a pH of 6.5 to 8.5, and b) acidic buffer has a pH of 1.5 to 3.

3. The HPLC based analytical method of claim 2 wherein the mobile phase comprises a gradient of three essential components a) basic buffer comprising ammonium phosphate, and having a pH of 7.7, b) acidic buffer comprising orthophosphoric acid and having a pH of 2.25, and c) acetonitrile.

4. The HPLC based analytical method of claim 1 wherein the mobile phase gradient over the run time of 100 min is as follows:

| Time (min) | Basic Buffer | Acidic Buffer | Organic Solvent |
|---|---|---|---|
| 0 | 100 | 0 | 0 |
| 15 | 100 | 0 | 0 |
| 40 | 10 | 80 | 10 |
| 65 | 0 | 82 | 18 |
| 70 | 0 | 82 | 18 |
| 80 | 0 | 65 | 35 |
| 90 | 65 | 0 | 35 |
| 100 | 100 | 0 | 0 |

5. The HPLC based analytical method of claim 1 wherein the flow rate of the mobile phase is 1 mL/min to 2 mL/min.

6. The HPLC based analytical method of claim 1 wherein the chromatographic detector is a PDA detector set over a range of 200-400 nm or a dual wavelength detector set at 210 and 300 nm.

7. The HPLC based analytical method of claim 1 wherein the column is Luna C-8(2), 5 μm (250 mm×4.6 mm).

8. Use of the HPLC based analytical method of claim 1 for preparing imipenem and cilastatin combination product of acceptable pharmaceutical purity.
